(19) United States
(12) Patent Application Publication
(10) Pub. No.: US 2010/0261686 A1
(43) Pub. Date: Oct. 14, 2010

(54) Pharmaceutical combination for the treatment of pain (acute or chronic), as powerful as a narcotic medication but non narcotic ,therefore very unlikely to create addiction

(76) Inventor: Jacques Benoit, Montreal (CA)

Correspondence Address: Jacques Benoit, 7013 Jean Milot, Montreal, QC, H1M3G4, CA

(21) Appl. No.: 12/384,881

(22) Filed: Apr. 10, 2009

(51) Int. Cl. A61K 31/167; A61K 31/60

(52) U.S. Cl. 514/162; 514/629

(57) ABSTRACT

The management of pain specially chronic pain often requires the use of opiate like medications which have a large number of undesirable effects, the most important one being habituation and addiction. The present invention consists of an integrated combination of medications acting through different mechanisms that are just as powerful as opiates in the management of pain without many of the undesirable side effects such as habituation and addiction.

### DESCRIPTION OF THE PREFERRED EMBODIMENTS

The embodiment of the invention in which an exclusive property or privilege is claimed are defined as follows

An integrated combination of medications acting through different mechanisms that are just as powerful as opiates in the management of pain without many of the undesirable side effects such as habituation and addiction.
1—Tramadol
2—Acetominophen OR any other standard non analgesic such as acetyl-salicylic acid (Aspirin)

3—Venlafaxine (Effexor) (SNRI) Serotonin Norepinephrine Reuptake inhibitors ( or any other SNRI Agent)
Or any Selective Serotonin Reuptake Inhibitors (SSRI)

Tramadol:

3 Mechanisms of Action
1—A centrally acting analgesic
2—MU opioid receptor agonist.
3—Noradrenaline reuptake inhibitor.
4—Serotonin reuptake inhibitor.
5—Acts on GABAergic, noradrenergic and serotonergic systems.

Adverse Effects: (Low Incidence)
Dizziness, nausea, constipation, somnolence, orthostatic hypotension, and rarely Physical or psychological dependence.

Acetaminophen:
1—Analgesic.
2—anti-pyretic.

Acetaminophen is one of the most popular and widely used drugs for the treatment of pain and fever. It occupies a unique position among analgesic drugs. Unlike NSAIDs it is almost unanimously considered to have no anti inflammatory activity and does not produce gastrointestinal damage or untoward cardio-renal effects. Unlike opiates it is almost ineffective in intense pain and has no depressant effect on respiration. Although acetaminophen has been used clinically for more than a century, its mode of action has been a mystery until about one year ago, when two independent groups (Zygmunt and colleagues and Bertolini and colleagues) produced experimental data unequivocally demonstrating that the analgesic effect of acetaminophen is due to the indirect activation of cannabinoid CB(1) receptors. In brain and spinal cord, acetaminophen, following deacetylation to its primary amine (p-aminophenol), is conjugated with arachidonic acid to form N-arachidonoylphenolamine, a compound already known (AM404) as an endogenous cannabinoid. The involved enzyme is fatty acid amide hydrolase. N-arachidonoylphenolamine is an agonist at TRPV1 receptors and an inhibitor of cellular anandamide uptake, which leads to increased levels of endogenous cannabinoids; moreover, it inhibits cyclooxygenases in the brain, albeit at concentrations that are probably not attainable with analgesic doses of acetaminophen. CB(1) receptor antagonist, at a dose level that completely prevents the analgesic activity of a selective CB(1) receptor agonist, completely prevents the analgesic activity of paracetamol. Thus, acetaminophen acts as a pro-drug, the active one being a cannabinoid. These findings finally explain the mechanism of action of acetaminophen and the peculiarity of its effects, including the behavioral ones. Curiously, just when the first CB(1) agonists are being introduced for pain treatment, it comes out that an indirect cannabino-mimetic had been extensively used (and sometimes overused) for more than a century.”

Further research has shown that paracetamol also modulates the endogenous cannabinoid system. Paracetamol is metabolized to AM404, a compound with several actions; most importantly, it inhibits the reuptake of endogenous cannabinoids and activates the nociceptor TRPV1. Either of these two actions by themselves has been shown to reduce pain, and both are a possible mechanism for paracetamol, though it has been demonstrated that after blocking cannabinoid receptors and hence making any action of cannabinoid reuptake irrelevant, paracetamol no longer has any analgesic effect, suggesting its pain-relieving action is indeed mediated by the endogenous cannabinoid system.

Acetominophen also acts by reducing production of prostaglandins, which are involved in the pain and fever processes, by inhibiting the cyclooxygenase (COX) enzyme. It thus acts as an antipyretic agent

Venlafaxine (Effexor) (SNRI)

3 Mechanisms of Action:
1—Analgesic
2—Antidepressant
3—Anxiokytic
(Considered a Non Specific Adjuvant)

Venlafaxine is a bicyclic antidepressant, and is usually categorized as a serotonin-norepinephrine reuptake inhibitor (SNRI), but it has been referred to as a serotonin-norepinephrine-dopamine reuptake inhibitor. It works by blocking the transporter “reuptake” proteins for key neurotransmitters affecting mood, thereby leaving more active neurotransmitter in the synapse. The neurotransmitters affected are serotonin (5-hydroxytryptamine) and norepinephrine (noradrenaline) Additionally, in high doses it weakly inhibits the reuptake of dopamine, with recent evidence showing that the norepinephrine transporter also transports some dopamine as well, implying that SNRIs may also increase dopamine transmission. This is because SNRIs work by inhibiting reuptake, i.e. preventing the serotonin and norepinephrine transporters from taking their respective neurotransmitters back to their storage vesicles for later use. If the norepinephrine transporter normally recycles some dopamine too, then SNRIs will also enhance dopaminergic transmission. Therefore, the antidepressant effects associated with increasing norepinephrine levels may also be partly or largely due to the concurrent increase in dopamine (particularly in the prefrontal cortex).

Suggested concentration for the combination medication (administered as tablet or capsule or liquid form such as a syrup).

Tramadol −30 MGs

Acetominophen 325 MG

Venlafaxine 10 MG

DOSAGE 1-2 TAB QID (1 to 2 tablets 4 times a day).

Clinically

10 patients suffering from chronic low back pain who were taking codeine at a dosage of 30 mgs 4 times a day were switched from codeine to the medication described in the present invention. 9 obtained a satisfactory control of their pain over a period of 6 months.

16 patients suffering from rheumatoid arthritis who were taking codeine at a dosage of 30 mgs 4 times a day were switched from codeine to the medication described in the present invention. 14 obtained a satisfactory control of their pain over a period of 12 months.

3 patients with acute pain from Zona (herpes zoster) obtained a satisfactory control of their pain.

16 patients suffering from chronic pain from fibromyalgia were treated with the medication described in the present invention. 12 obtained a satisfactory control of their pain over a period of 6 months.

### SUMMARY OF THE INVENTION

the present invention describes an integrated combination of medication that can be used for the control of chronic or acute pain of various origins with the same efficacy as opiate medication and without the side effects of opiates. The combination of Tramadol −30 Mgs Acetominophen 325 MG and Venlafaxine 10 MG taken as 1-2 tablet 4 times a day proved effective in clinical situations either when the present treatment was substituted for an opiate medication or when the present treatment was started on a patient de novo.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims herein made.

1- A pharmaceutical combination comprising:
1—Tramadol
2—Acetominophen OR any other standard analgesic such as acetyl-salicylic acid (Aspirin) or non steroid anti inflammatory agent (nsaid)
3—Venlafaxine (Effexor) (SNRI) Serotonin Norepinephrine Reuptake inhibitors ( or any other SNRI Agent)
Or any Selective Serotonin Reuptake Inhibitors (SSRI)

2- This integrated medication can be taken orally

3- This integrated medication can be taken parenterally by intravenous or intramuscular injection.

4- This medication can be taken using a transcutaneous patch.
